(19) United States
(12) Patent Application Publication
(10) Pub. No.: US 2017/0367676 A1
(43) Pub. Date: Dec. 28, 2017

(54) SYSTEM FOR DETECTING DISEASE OF THE INTERNAL ORGANS FROM VOICE, WAVEFORM AND PHYSIOLOGICAL CHANGES

(71) Applicant: MEDVOICE BIOTECH CORP., Fountain Valley, CA (US)

(72) Inventors: Bia Mac, Irvine, CA (US); Theresa Quach, Irvine, CA (US); Leo Lu, Garden Grove, CA (US); Thuan Hoang, Tan Binh Distr, HCM City (VN); Calvin Cao, Costa Mesa, CA (US); Lam Le, Ho Chi Minh City (VN); Thomas Vo, Anaheim, CA (US)

(21) Appl. No.: 15/190,660

(22) Filed: Jun. 23, 2016

(51) Int. Cl. A61B 7/02; A61B 5/0205; A61B 5/00; A61B 5/024; A61B 5/1455

(52) U.S. Cl. CPC A61B 7/02; A61B 5/0205; A61B 5/742; A61B 5/0024; A61B 5/14551; A61B 5/024

(57) ABSTRACT

Audio analysis is combined with physiological data to provide indications of the health of bodily organs and systems.

### BACKGROUND OF THE INVENTION

The present invention is related to the medical field, and in particular to a method and apparatus for detecting disease from physiological data to include audio frequency changes.

There are many existing patents for detecting heart conditions such as heart beat rate and oxygen levels in the blood. Such inventions, however, do not address other internal organs in the body that contribute an equally important part of the overall health of the human.

Thus, there is a need for a new methodology and system for evaluating the condition of internal organs from voice, waveform, and physiological changed parameters by the use of smart non-invasive mobile device sensors, that provides interpretations within seconds.

### DESCRIPTION OF RELATED ART

U.S. Pat. No. 8,827,906 discloses a method, system and device for measuring fingertip heart rate using motion sensors, a light source and a light detector.

U.S. Pat. No. 5,735,285 discloses using a handheld device to pick up ECG signals for heart rate. Similarly, U.S. Pat. No. 6,254,614 discloses a heart monitor via audible signal outputs.

U.S. Pat. No. 6,685,633 discloses a heart monitor that a patient can hold against the chest.

U.S. Pat. No. 6,820,057 discloses a system to acquire, record and transmit ECG data regarding the heart rate and pulse monitor.

U.S. Pat. No. 8,948,832 discloses a wearable heart rate monitor with a motion and photoplethysmographic sensor.

US Patent Publication 2014/0235980 discloses a method, devices and a system for measuring and monitoring heart rate and pulse.

U.S. Pat. No. 8,373,787 discloses capturing and analyzing EKG data from a user, using a smartphone.

U.S. Pat. No. 7,222,075 discloses a system, method to detect emotional states using through analysis of a user's voice.

WO Publication PCT/US2013/064041 discloses that neurological disease such as Parkinson's disease can be detected from a user's speech based on human factor cepstral coefficients (HFCC). The calculated result is based on the articulation range and articulation rate of the speech. This is only significant with medical conditions that affect the speech aspect of the user like Parkinson's disease.

U.S. Pat. No. 9,198,613 discloses an apparatus and method of diagnosing health by calculating the change of voice due to the occipital part of the brain and/or the change of the occipital bone that controls the voice, thus giving rise to voice differences. Otherwise, the brain—without the occipital portion problem—cannot detect the health problem.

The above representative prior art discloses measuring and monitoring the condition of the heart along with blood oxygen level, but is unable to evaluate the condition of other main internal organs, which play a crucial part of the overall health of the user.

### SUMMARY OF THE INVENTION

The present invention provides a method, system and device for detecting disease of the internal organs and related body systems by analyzing voice, waveform and physiological parameter changes. Signals are captured by biosensors (either attached to the body or wireless). These signals are processed and compared to a reference data. The results are displayed on a screen along with options to connect to local healthcare providers or vendors and can be stored online or temporarily offline for future reading. At least one input is voice data.

The invention provides a mobile, non-invasive method which can provide well-being interpretations within seconds. Early detection is best for preventive health and provides a beneficial approach to generate awareness for healthy living.

The present invention also provides a way of revealing the interrelationship of the different body system conditions as well as the main internal organs.

### BRIEF DESCRIPTION OF THE DRAWINGS

FIG. 1 is a flow chart of the data process according to the present invention.

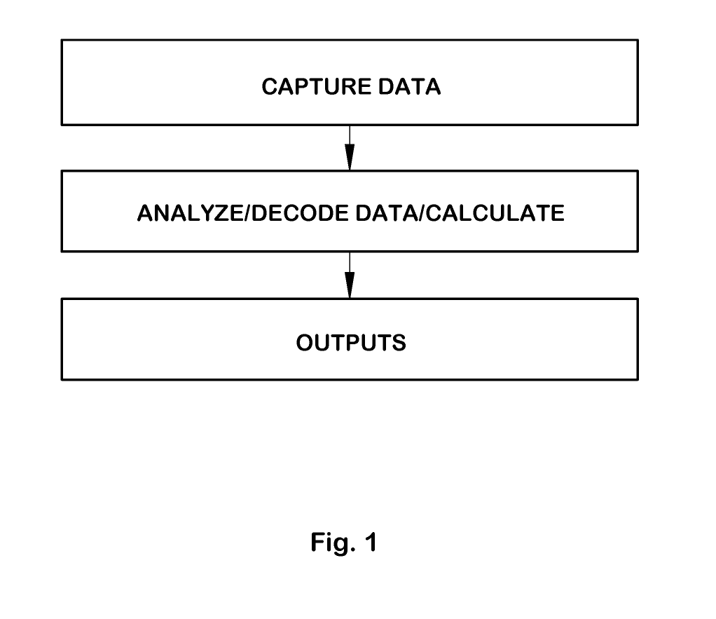

FIG. 2 is a flow chart of a process according to one embodiment of the invention.

### DETAILED DESCRIPTION OF THE INVENTION

Beside the user's heart rate and pulse, blood oxygen level is vital to one's health. This level can be measured with an oximeter, as described in many prior patents.

The condition of the main internal organs can be determined with this invention, by analyzing captured data of the user's voice parameters and/or waveforms together with physiological changes. At any time, abnormalities or malfunctions of one or more internal organs may affect the user's overall health; such abnormalities or malfunctions may show in the voice, waveform, and/or in changed physiological parameters.

An EKG provides a waveform that is unique to the heart. Similarly, other internal organs give out unique waveforms.

By comparing normal parameters to one and/or more waveforms from the internal organs, one can determine the systemic disharmonies of the overall health. For example, the digestive system includes the spleen, stomach, liver, gall bladder, etc.

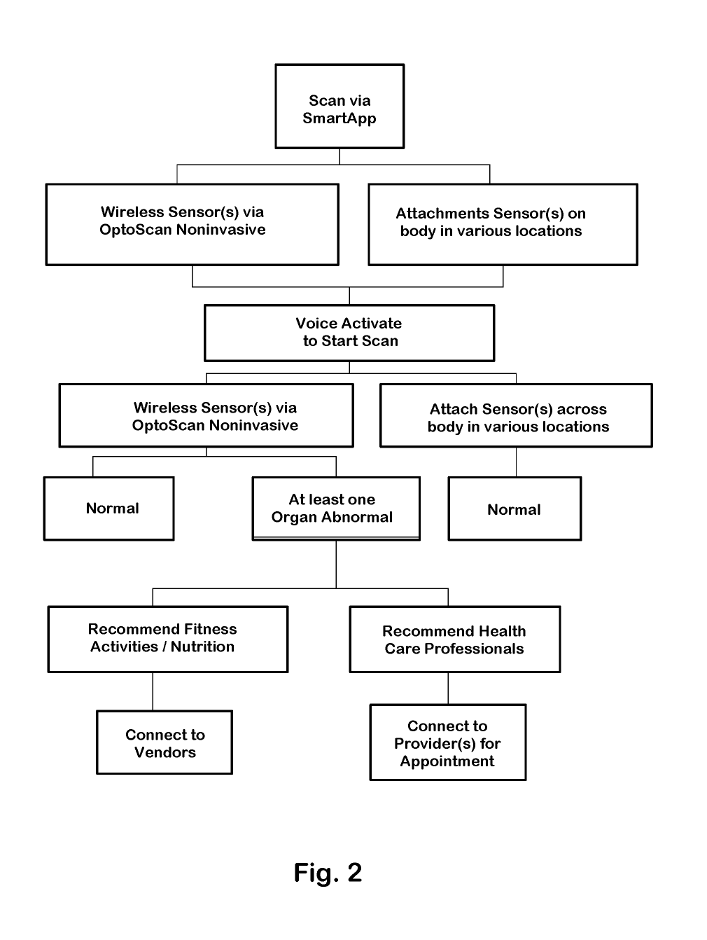

As shown in FIG. 2, a body scan is activated by a smart app, which activates noninvasive wireless sensors and contact sensors which are attached to the body. Voice activation starts the scan, and a sample of the voice is retained for analysis.

The results captured by the sensor, and the voice sample, are then compared by a computed algorithm with prior or reference data, and the actual differences are displayed.

After the detection of the possible conditions of the internal organs, preventive measures in terms of description, recommended physical activities, nutrition and lifestyle awareness are presented.

Connection and contact information of local healthcare professionals are provided as options, as well as local vendors for health products.

Optionally, captured and calculated data may be retained to improve overall accuracy.

The user may opt in to create a person health profile on the inventor's virtual storage server for open collaboration to learn from and share with a community of peers and care professionals.

Inasmuch as the invention is subject to variations and modifications, it is intended that the foregoing shall be interpreted as merely illustrative of the invention defined by the following claims.

We claim:

1. A diagnostic method comprising:
capturing input data including an audio sample and at least one other input indicative of a physiological parameter or waveform,
calculating, from said audio sample and said at least one other input, results indicative of a disorder or malfunction of at least one organ or body system, and
displaying results of said calculating on a screen.

2. The method of claim 1, wherein said parameters measured at locations of the body including at least one of the wrist, finger, neck, face, groin, ankle, torso left side, and torso right side.

3. The method of claim 1, wherein said parameters are measured by devices selected from the group consisting of attached sensors, wireless sensors, microphones and cameras.

4. The method of claim 1, further comprising a step of adjusting capacities for picking up signals at least 1-60 seconds time frame and the focused area within ideal proximity with adjusting visual cues.

5. The method in claim 1, wherein said body system is selected from the group consisting of:
(a) cardiovascular system/circulation system
(b) digestive/excretory system
(c) skeletal system
(d) muscular system
(e) respiratory system
(f) nervous system
(g) endocrine system
(h) reproductive system
(I) renal/urinary system
(j) immune system
(k) lymphatic system and
(l) integumentary/exocrine system.

6. The method of claim 1, wherein said at least one organ is selected from the group consisting of lung, large intestine, heart, small intestine, spleen, stomach, liver, gall bladder, kidney and urinary bladder.

7. The method of claim 1, wherein said captured data is stored for future use.

8. The method of claim 1, where the condition of said at least one organ is continuously monitored for the purpose of stabilizing vital signs and warning of possible failure.

9. The method of claim 1, wherein at least one said other input is pulse data.

10. The method of claim 1, wherein at least one said other input is oximeter data.

11. The method of claim 10, wherein said oximeter data is obtained by processing a video recording of the wrist.

12. A diagnostic system comprising
a microphone for obtaining a sample of a subject's voice,
at least one other sensor for obtaining input data representative of a physical parameter or waveform of the body,
a processor for calculating, from said voice sample and said at input data, results indicative of a disorder or malfunction of at least one organ or body system, and
a screen for displaying said results.

13. The system of claim 12, wherein said sensors are placed on the body at locations including the wrist, finger, neck, face, groin, ankle, torso left side, torso right side.

14. The system of claim 12, wherein said at least one other sensor is selected from the group consisting of attached sensors, wireless sensors, microphones and cameras.

15. The system of claim 12, further comprising means for storing at least one of said voice sample, said input data and said calculated results.
